(19) United States
(12) Patent Application Publication
(10) Pub. No.: US 2024/0306512 A1
(43) Pub. Date: Sep. 12, 2024

(54) Multi-Layer Flexible Array Interconnect for an Ultrasound Transducer, and Methods of Manufacture

(71) Applicant: FUJIFILM SonoSite, Inc., Bothell, WA (US)

(72) Inventors: Andrew Lundberg, Woodinville, WA (US); Jimin Zhang, Bellevue, WA (US); William Blakemore, Bothell, WA (US); Ken Best, Monroe, WA (US)

(73) Assignee: FUJIFILM SonoSite, Inc., Bothell, WA (US)

(21) Appl. No.: 18/591,845

(22) Filed: Feb. 29, 2024

Related U.S. Application Data
(60) Provisional application No. 63/489,369, filed on Mar. 9, 2023.

(51) Int. Cl. H10N 30/87; A61B 8/00; B06B 1/06; H10N 30/06; H10N 30/088

(52) U.S. Cl. CPC H10N 30/875; A61B 8/4494; B06B 1/067; H10N 30/06; H10N 30/088; B06B 2201/76

(57) ABSTRACT

Systems and methods for a multi-layer flexible array interconnect for an ultrasound transducer, and methods of manufacture are disclosed. The systems and methods described herein provide consistent and controllable alignment between the odd and even flex layers. Further, ground layers are added between the signal layers to improve crosstalk performance while simplifying the construction. In some implementations, vias are introduced at the interface of an electromechanical-array element (e.g., lead zirconate titanate (PZT) element) to increase the surface area of conductive material and thereby increase the quality of the electrical and physical connections. In addition, a machining alignment and verification aid is provided through strategic use of unused and discarded board area to enable (i) alignment check of the flex circuit with the machining equipment and (ii) adjustment of the flex circuit position relative to the machining equipment to avoid the flex circuit being machined to an unusable state.

### CROSS-REFERENCE TO RELATED APPLICATIONS

This application claims priority to U.S. Provisional Application Ser. No. 63/489,369, filed Mar. 9, 2023, the disclosure of which is incorporated herein by reference in its entirety.

### BACKGROUND

Ultrasound scanners (e.g., probes) are extremely sensitive devices. Manufacture of ultrasound transducer arrays, which are central to scanner performance, is challenging due to various factors such as the very small size of the circuit elements and tolerance limits, the large number of connections, durability requirements of the use environment and high-performance expectations. Physical limitations in the manufacturing process limit desired design parameters.

One partial solution for transducer array construction is described in U.S. Pat. No. 6,043,590, filed in 1997 and titled “Composite Transducer with Connective Backing Block,” which demonstrates a method of imbedding a flex circuit in a backing block in a way that creates a durable interconnect to the lead zirconate titanate (PZT) crystal. That method has been further enhanced by another approach described in U.S. Pat. No. 7,804,970, filed in 2005 and titled “Array Interconnect for Improving Directivity,” which enhances the performance of the array by using two flex circuits split into odd and even elements. This odd/even approach reduces crosstalk between neighboring elements, which in turn broadens the element pattern acceptance angle and improves the quality of beamforming.

One challenge of this odd/even type of array construction is ensuring that a good connection is made between the end of the trace on the flex circuit (contact surface area defined by the width and thickness of a copper trace) and the plated PZT crystal. As the performance of new transducers advances, the element pitch and geometry gets smaller and therefore much more challenging to produce. As a result, the transducer yields are decreased due to open and short circuits associated with the imbedded flex array construction.

In addition to the small amount of contact surface area presented by the end of the copper trace, the physical registration between the odd and even flex circuits is challenging the limits of trace width and spacing as the odd and even flex circuits approach the pitch of the arrays.

### SUMMARY

Systems and methods for a multi-layer flexible array interconnect for an ultrasound transducer, and methods of manufacture are disclosed. The systems and methods disclosed herein provide more-consistent and more-controllable alignment between odd and even flex layers of an array interconnect. Further, ground layers are added between the signal layers to improve crosstalk performance while simplifying construction of the array. In some implementations, vias are introduced at the interface of the electromechanical-array element (e.g., PZT element) to increase the surface area of conductive material and thereby increase the quality of the electrical and physical connections. In addition, a machining alignment and verification aid is provided through strategic use of unused and discarded board area to enable (i) alignment check of the flex circuit with the machining equipment and (ii) adjustment of the flex circuit position relative to the machining equipment to avoid the flex circuit being machined to an unusable state (e.g., cut beyond a tolerance).

In aspects, a flex array interconnect for an ultrasound circuit is disclosed. The flex array interconnect includes a first signal layer, a second signal layer, and one or more ground layers. The first signal layer includes a first plurality of conductive traces configured to be electrically and physically connected to an interface of an electromechanical-array element of an ultrasound transducer. The second signal layer includes a second plurality of conductive traces configured to be electrically and physically connected to the interface of the electromechanical-array element of the ultrasound transducer. The one or more ground layers are disposed between the first and second signals layers to reduce crosstalk between the first and second signal layers.

In aspects, another flex array interconnect for an ultrasound circuit is disclosed. The flex array interconnect includes a plurality of layers and a plurality of vias. The plurality of layers include at least a first layer and a second layer, where the first layer has a first plurality of conductive traces and the second layer has a second plurality of conductive traces. The plurality of vias are filled and plated with conductive material. A via of the plurality of vias connects a trace of the first plurality of traces in the first layer to a third layer of the plurality of layers. The via provides an enlarged surface area for the trace at a machining interface of the flex array interconnect, in comparison to a cross section of the trace, for connection to an interface of an electromechanical-array element of an ultrasound transducer.

In aspects, yet another flex array interconnect for an ultrasound circuit is disclosed. The flex array interconnect includes a first layer and a second layer. The first layer includes a first plurality of conductive traces comprising a first set of machining alignment aids. The second layer is stacked with the first layer and has a second plurality of conductive traces comprising a second set of machining alignment aids. The first and second sets of machining alignment aids provide measurable indicators of machining depth during the machining process.

In aspects, a method is disclosed. The method includes casting a backing block from epoxy with embedded flex circuits including one or more machining alignment aids connected to signal traces on the embedded flex circuits. The method also includes machining the backing block to a correct geometry to expose the signal traces on the flex circuits for interfacing with an electromechanical array. In addition, the method includes during the machining of the backing block, monitoring the machining based on the one or more machining alignment aids of the embedded flex circuits of the backing block. Further, the method includes verifying, based on the monitoring, an alignment of the backing block with the machining device based on the one or more machining alignment aids of the embedded flex circuits. Also, the method includes, after the backing block is machined to the correct geometry, bonding the electromechanical array to the backing block to form a bonded module. Additionally, the method includes placing the bonded module in a dicing saw and dicing the bonded module with a dicing saw to cut each element of the bonded module to a depth that isolates each element and a corresponding electrical connection.

### BRIEF DESCRIPTION OF THE DRAWINGS

The appended drawings illustrate examples and are, therefore, exemplary implementations and not considered to be limiting in scope.

FIG. 1 illustrates an example environment for an ultrasound system having an ultrasound scanner, in accordance with one or more implementations.

FIG. 2 illustrates an example implementation of the ultrasound scanner from FIG. 1.

FIG. 3 illustrates a cross-section of an example array architecture and interconnection for a high-sensitivity transducer of an ultrasound scanner.

FIG. 4A illustrates an implementation of a single flex circuit.

FIG. 4B illustrates an implementation of a dual flex circuit.

FIG. 5 illustrates and example implementation of a dual flex circuit with a ground plane.

FIG. 6 illustrates an example implementation of a multi-layer flex circuit with internal ground plane.

FIG. 7 illustrates and example implementation of a multi-layer flex circuit with internal and external ground planes.

FIG. 8 illustrates an example implementation of a multi-layer flex circuit with internal ground planes and plated and filled micro-vias at a machining interface.

FIG. 9 illustrates an example implementation of a multi-layer flex circuit with internal and external ground planes and plated and filled micro-vias at the machining interface.

FIG. 10 illustrates an example implementation of a multi-layer flex circuit with internal ground planes and plated and filled micro-vias below the machining interface.

FIG. 11 illustrates an example implementation of a multi-layer flex circuit with internal and external ground planes and plated and filled micro-vias below the machining interface.

FIG. 12 illustrates an example implementation of a multi-layer flex circuit with internal and external ground planes and plated and filled through-hole vias at the machining interface.

FIG. 13 illustrates an example multi-layer flex circuit embedded in a backing block.

FIG. 14 illustrates an enlarged view of a section from FIG. 13.

FIG. 15 illustrates an example flex circuit with additional features providing machining alignment aid.

FIG. 16 illustrate an example implementation of a multi-layer flex circuit having multiple machining alignment aids.

FIG. 17 illustrates another view of the multi-layer flex circuit from FIG. 16.

FIG. 18 depicts a method of manufacturing a flex circuit in accordance with the implementations disclosed herein.

### DETAILED DESCRIPTION

Systems and methods for a multi-layer flexible array interconnect for an ultrasound transducer, and methods of manufacture are disclosed. The systems and methods disclosed herein provide more-consistent and more-controllable alignment between the odd and even flex layers. For example, during the process of casting a backing block of the transducer, constraints of flex circuit construction methods are relied on instead of mechanical fixturing. Further, ground layers are added between the signal layers to improve crosstalk performance while simplifying construction of the array by reducing the number of flex circuits used in the ultrasound transducer. In some implementations, vias are introduced at the electromechanical-array interface (e.g., PZT interface). The presence and location of these vias increases the surface area of conductive material and thereby increases the quality of the electrical and physical connections.

In addition, a machining alignment and verification aid is provided through strategic use of unused and discarded board area. This machining alignment and verification aid enables (i) alignment check of the flex circuit with the machining equipment during the machining process and (ii) adjustment of the flex circuit position relative to the machining equipment to avoid the flex circuit being machined to an unusable state (e.g., cut to a state in which the copper trace of the flex circuit cannot be aligned to electrically connect with the plated PZT crystal). The machining alignment and verification aid includes markers located in a portion of the board being machined away to provide an indication of the alignment as well as cut depth of the flex circuit relative to the machining equipment.

### Ultrasound System

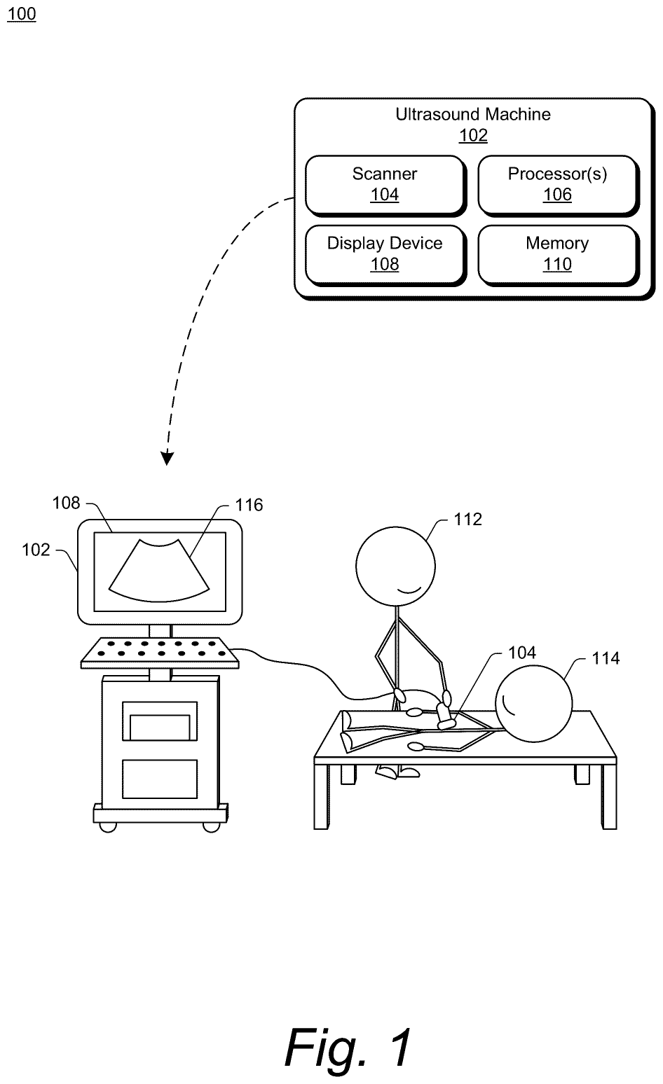

FIG. 1 illustrates an example environment for an ultrasound system 100 having an ultrasound scanner, in accordance with one or more implementations. Generally, the ultrasound system 100 includes an ultrasound machine 102, which generates data based on high-frequency sound waves reflecting off body structures. The ultrasound machine 102 includes various components, some of which include a scanner 104, one or more processors 106, a display device 108, and a memory 110.

A user 112 (e.g., nurse, ultrasound technician, operator, sonographer, etc.) directs the scanner 104 toward a patient 114 to non-invasively scan internal bodily structures (organs, tissues, etc.) of the patient 114 for testing, diagnostic, or therapeutic reasons. In some implementations, the scanner 104 includes an ultrasound transducer array and electronics communicatively coupled to the ultrasound transducer array to transmit ultrasound signals to the patient's anatomy and receive ultrasound signals reflected from the patient's anatomy. In some implementations, the scanner 104 is an ultrasound scanner, which can also be referred to as an ultrasound probe.

The display device 108 is coupled to the processor 106, which processes the reflected ultrasound signals to generate ultrasound data. The display device 108 is configured to generate and display an ultrasound image (e.g., ultrasound image 116) of the anatomy based on the ultrasound data generated by the processor 106 from the reflected ultrasound signals detected by the scanner 104. In some aspects, the ultrasound data includes the ultrasound image 116 or data representing the ultrasound image 116.

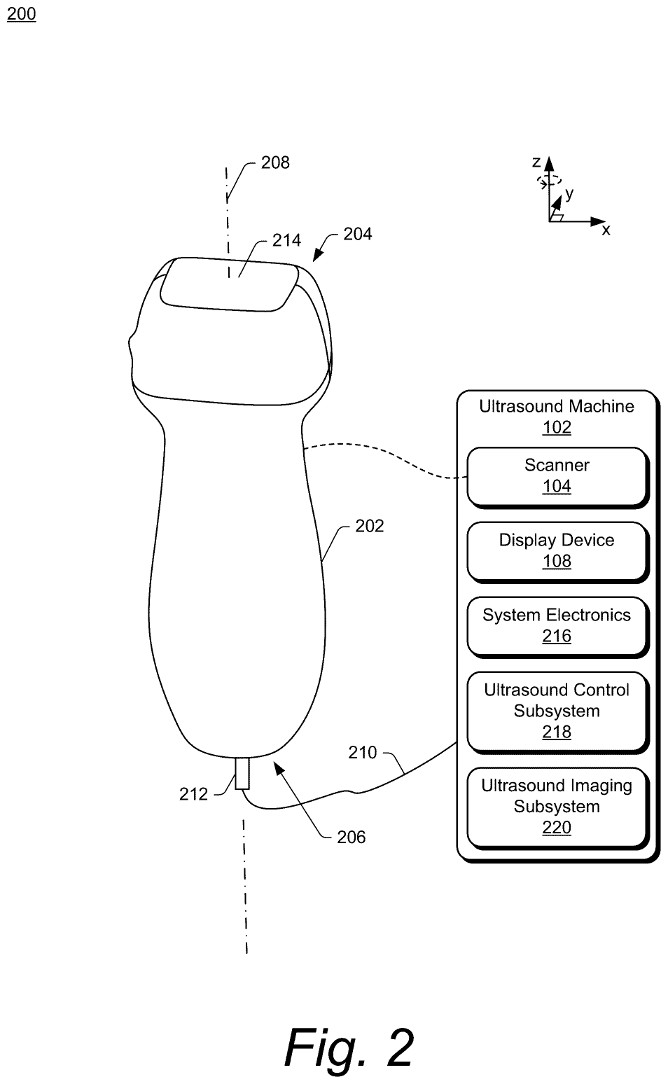

FIG. 2 illustrates an example implementation 200 of the ultrasound scanner 104 from FIG. 1. The scanner 104 (e.g., ultrasound scanner) includes an enclosure 202 extending between a distal end portion 204 and a proximal end portion 206. The enclosure 202 includes a central axis 208 (e.g., longitudinal axis) that intersects the distal end portion 204 and the proximal end portion 206. The scanner 104 is electrically coupled to an ultrasound imaging system (e.g., the ultrasound machine 102) via a cable 210 that is attached to the proximal end portion 206 of the scanner 104 by a strain relieve element 212. In some implementations, the scanner 104 is wirelessly coupled to the ultrasound imaging system and communicates with the ultrasound imaging system via one or more wireless transmitters, receivers, or transceivers over a wireless connection or network (Bluetooth™, Wi-Fi™, etc.).

A transducer assembly 214 having one or more transducer elements is electrically coupled to system electronics 216 in the ultrasound machine 102. In operation, the transducer assembly 214 transmits ultrasound energy from the one or more transducer elements toward a subject and receives ultrasound echoes from the subject. The ultrasound echoes are converted into electrical signals by the transducer element(s) and electrically transmitted to the system electronics 216 in the ultrasound machine 102 for processing and generation of one or more ultrasound images.

Capturing ultrasound data from a subject using a transducer assembly (e.g., the transducer assembly 214) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than 15 Megahertz (MHz)) and/or high-frequency ultrasound (e.g., greater than or equal to 15 MHz). A particular frequency range to use can readily be determined based on various factors, including, for example, depth of imaging, desired resolution, and so forth.

In some implementations, the system electronics 216 include one or more processors (e.g., the processor(s) 106 from FIG. 1), integrated circuits, application-specific integrated circuits (ASICs), Field Programmable Gate Arrays (FPGAs), and power sources to support functioning of the ultrasound machine 102. In some implementations, the ultrasound machine 102 also includes an ultrasound control subsystem 218 having one or more processors. At least one processor, FPGA, or ASIC causes electrical signals to be transmitted to the transducer(s) of the scanner 104 to emit sound waves and receives electrical pulses from the scanner 104 that were created from the returning echoes. One or more processors, FPGAs, or ASICs process the raw data associated with the received electrical pulses and form an image that is sent to an ultrasound imaging subsystem 220, which causes the image (e.g., the image 116 in FIG. 1) to be displayed via the display device 108. Thus, the display device 108 displays ultrasound images from the ultrasound data processed by the processor(s) of the ultrasound control subsystem 218.

In some implementations, the ultrasound machine 102 also includes one or more user input devices (e.g., a keyboard, a cursor control device, a microphone, a camera, etc.) that input data and enable taking measurements from the display device 108 of the ultrasound machine 102. The ultrasound machine 102 can also include a disk storage device (e.g., computer-readable storage medium such as read-only memory (ROM), a Flash memory, a dynamic random-access memory (DRAM), a NOR memory, a static random-access memory (SRAM), a NAND memory, and so on) for storing the acquired ultrasound images. In addition, the ultrasound machine 102 can include a printer that prints the image from the displayed data. To avoid obscuring the techniques described herein, such user input devices, disk storage device, and printer are not shown in FIG. 2.

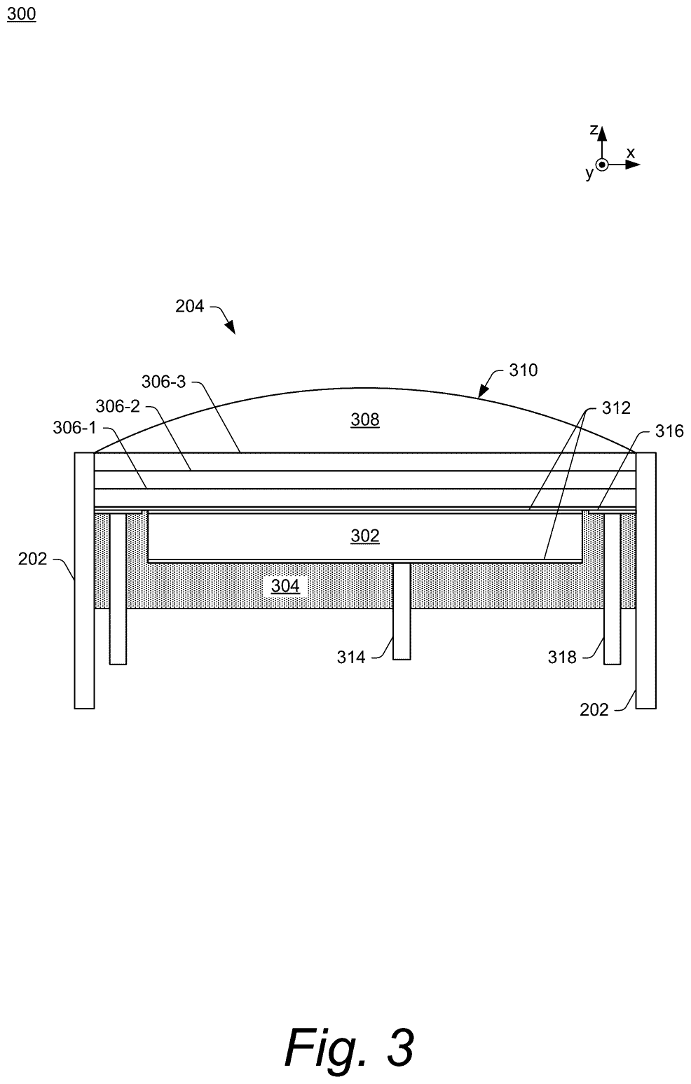

FIG. 3 illustrates a cross-section 300 of an example array architecture and interconnection for a high-sensitivity transducer of an ultrasound scanner. The cross-section 300 represents a cross-section of the distal end portion 204 of the ultrasound scanner 104 in FIG. 2.

In the illustrated example, the ultrasound scanner (e.g., the ultrasound scanner 104 of FIG. 2) includes transducer piezoelectric ceramic elements (e.g., piezoelectric material 302) sandwiched between a backing material 304 and a set of matching layers 306 (e.g., matching layers 306-1, 306-2, and 306-3). On the side of the set of matching layers 306 opposite from the piezoelectric material 302 is an acoustic lens (e.g., lens 308), which has an outer surface 310 facing outward (away from the proximal end portion 206 shown in FIG. 2). The outer surface 310 of the lens 308 is convex (e.g., curved toward the set of matching layers 306).

The piezoelectric material 302 includes crystals with electrodes (e.g., electrodes 312) formed by, for example, plating a thin film of gold or silver on the crystal surface. The piezoelectric material 302 can be made of any suitable material, an example of which is lead zirconate titanate (PZT), though others include quartz, barium titanate, and polyvinylidene fluoride (PVDF). The electrodes 312 can include a first electrode between the piezoelectric material 302 and the set of matching layers 306 and a second electrode between the piezoelectric material 302 and the backing material 304. In the illustrated example, the first electrode is adjacent to the front side of the piezoelectric material 302 (e.g., facing the lens 308) and the second electrode is adjacent to the backside of the piezoelectric material 302 (e.g., facing the backing material 304). The set of matching layers 306 provides an acoustic impedance gradient for acoustic energy generated by the piezoelectric material 302 to smoothly penetrate the body tissue of the patient and for the reflected acoustic waves (the returning echo) to smoothly return to the piezoelectric material 302 for detection.

In some implementations, the backing material 304 has a U-shaped cross-section having a recessed area for receiving the piezoelectric material 302. In this way, the backing material 304 provides structural support on the backside (away from the lens 308) as well as lateral sides (sides intersecting the x-axis and/or the y-axis) of the piezoelectric material 302. Accordingly, a portion of the backing material 304 is between the piezoelectric material 302 and the enclosure 202 (e.g., in the x- and y-directions). The backing material 304 can be made of various materials. Some examples include tungsten, iron, magnesium, and aluminum. In some implementations, the backing material 304 is a composite support material including, for example, an epoxy joined with tungsten particles. The backing material 304 is configured to prevent backward emitted sound waves from echoing and ringing back into the piezoelectric material 302 for detection.

The ultrasound scanner 104 also includes an electrical connection 314 to a power supply to provide alternating current to the second electrode. In addition, a ground electrode 316 provides an electrical connection to ground 318 and serves as a terminal of the backside of the piezoelectric material 302.

The example illustrated in FIG. 3 is one example of the array architecture and interconnection for the transducer of the ultrasound scanner 104. The techniques disclosed herein can also be implemented using other types of ultrasound transducers, which include other configurations of array architectures and interconnections. For example, the techniques disclosed herein can be applied using a piezoelectric micromachined ultrasonic transducer (PMUT), a capacitive micromachined ultrasonic transducer (CMUT), or any other suitable electromechanical array, sensor, or transducer, which uses an electrical connection from structure to another structure.

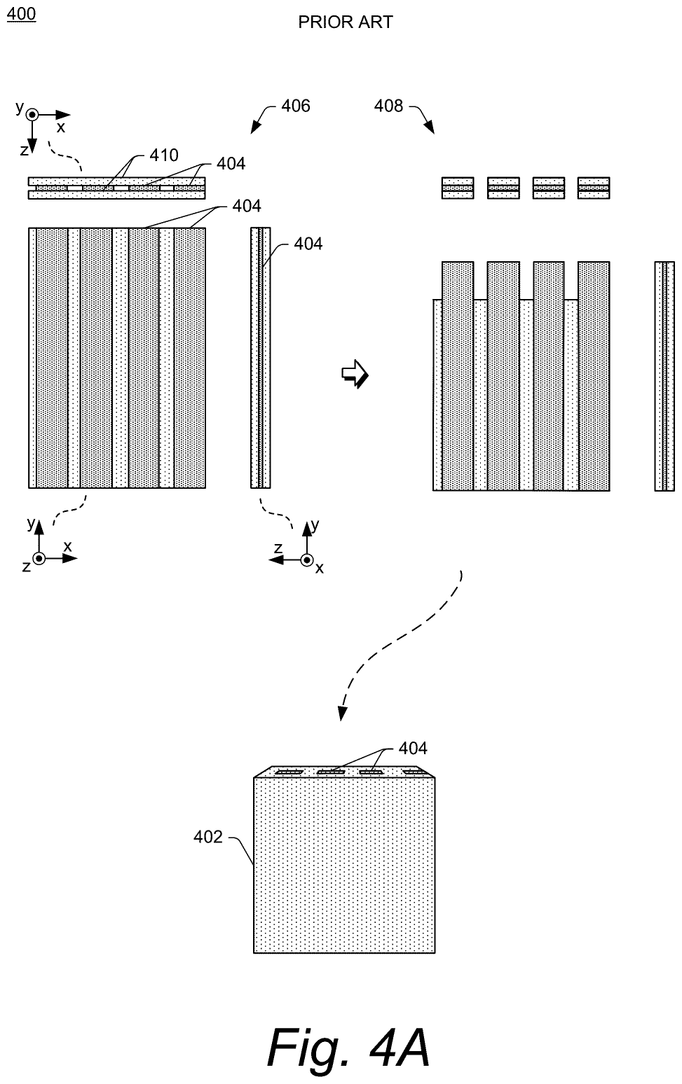

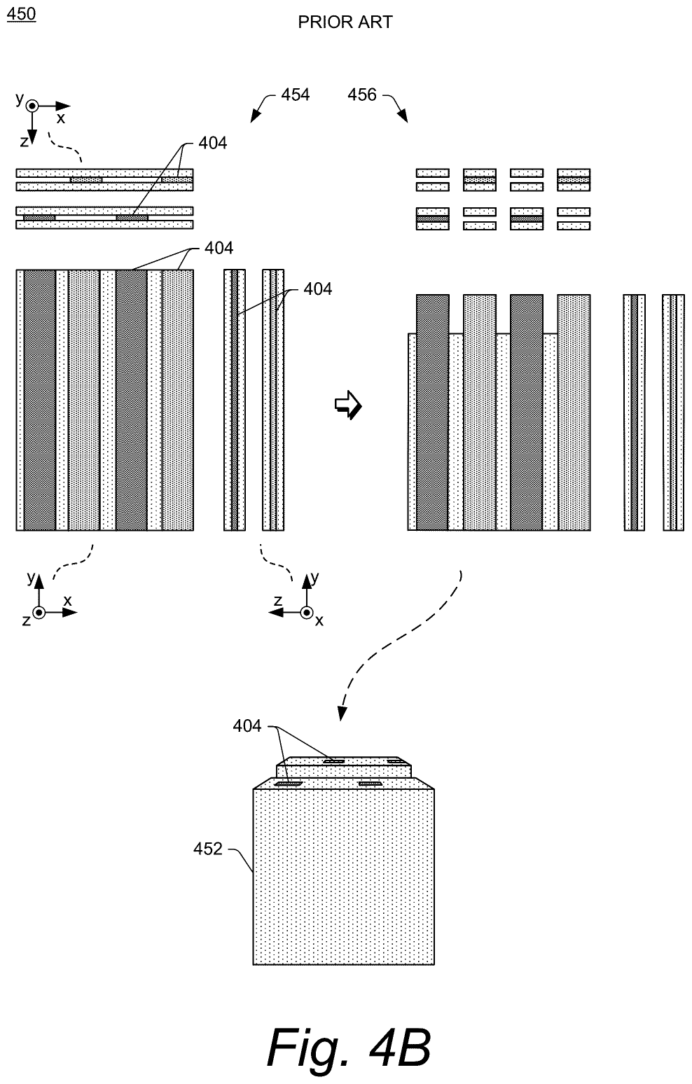

FIGS. 4A through 12 illustrate example flex circuits used for ultrasound transducers. Generally, when constructing an ultrasound array module, a backing block is cast from epoxy with embedded flex circuits. Once the backing block is set, it is machined to the correct geometry to interface with the PZT array and expose the signal traces on the flex circuits for connection to the PZT. Then, the PZT is bonded to the backing block. Next, this bonded module is placed in a dicing saw where each “element” is cut to a depth that isolates each element and its electrical connection. In each of FIGS. 4A through 12, each circuit is shown with a front view (xy-plane), a corresponding right view (yz-plane), and top view (xz-plane).

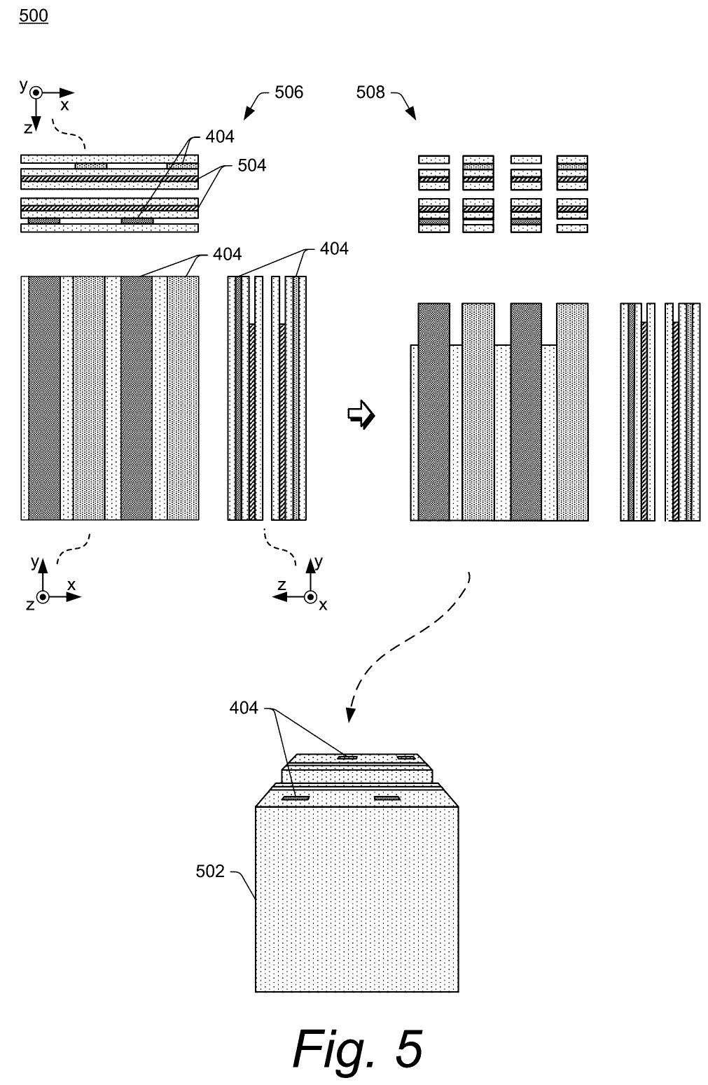

In historical implementations with single and dual flex circuits, shown in FIGS. 4A and 4B, respectively, special tooling is required to ensure the alignment of the flex circuits while they are cast in epoxy and then machined and diced. If the fixturing is not adequate, the odd and even traces can overlap and cause shorts and opens while dicing. However, using a multi-layer flex (with three or more layers), shown in FIGS. 5 through 12, enables registration of the odd and even signal layers to tighter tolerances, respectively, which reduces the complexity of the tooling alignment.

As mentioned, FIG. 4A illustrates an implementation 400 of a single flex circuit 402. For simplicity, the single flex circuit 402 is illustrated to show conductive (e.g., copper) traces 404 prior to dicing (at 406) and after dicing (at 408). Each trace 404 is adjacent to an insulating layer 410 on each side of the trace 404 (in a direction of the z-axis), such that the trace 404 is between two adjacent insulating layers 410. FIG. 4B illustrates an implementation 450 of a dual flex circuit 452. The dual flex circuit 452 is illustrated to show the conductive traces 404 prior to dicing (at 454) and after dicing (at 456).

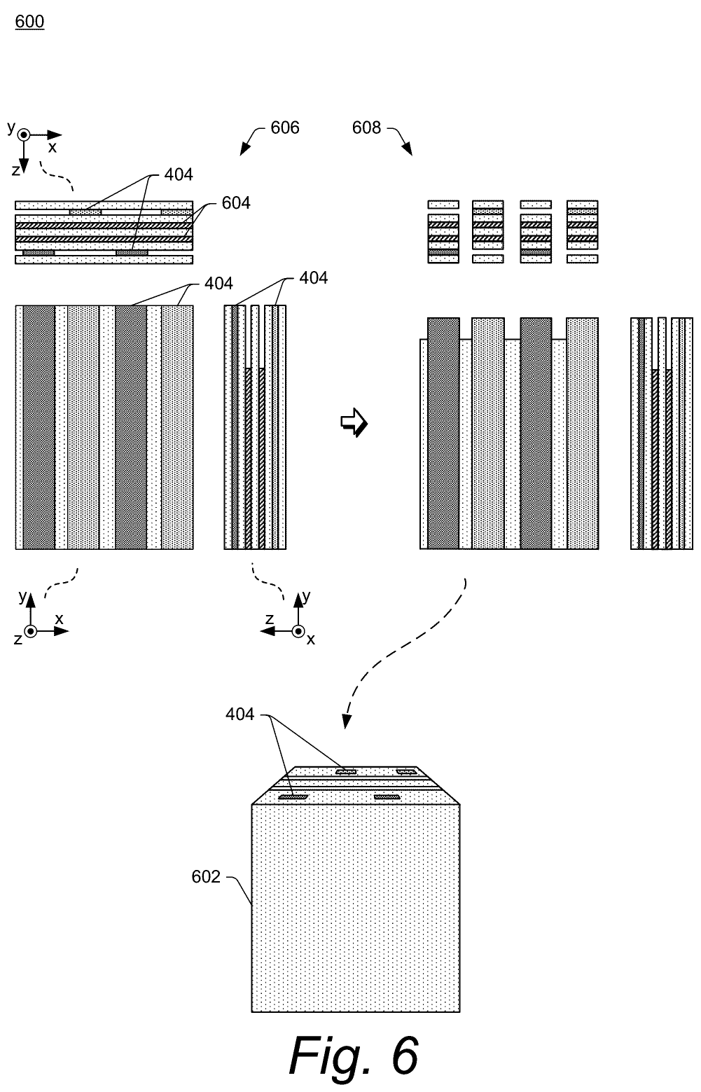

Using the dual flex circuit 452 (in FIG. 4B) reduces “element-to-element” crosstalk introduced from neighboring traces on the flex circuit compared to the signal flex circuit 402 (in FIG. 4A) by increasing the signal trace spacing on the flex circuit. The result of this improvement can be seen as improved element patterns and yields a higher-performing transducer. If the dual flex circuit is implemented with odd and even flex circuits having no spacing between them, the crosstalk would increase, approaching that of a single flex circuit. Adding a ground plane to the flex circuit can mitigate this issue and enable greater freedom in the array block design and fixturing. This ground plane can be implemented with a dual flex design (FIG. 5) or with a multilayer flex with both odd and even traces (e.g., FIGS. 6 to 9).

FIG. 5 illustrates and example implementation 500 of a dual flex circuit 502 with a ground plane 504. For simplicity, the dual flex circuit 502 is illustrated to show the traces 404 prior to dicing (at 506) and after dicing (at 508).

FIG. 6 illustrates and example implementation 600 of a multi-layer flex circuit 602 with an internal ground plane 604 (e.g., internal ground layer). In aspects, the internal ground plane 604 can include two layers of ground. For simplicity, the multi-layer flex circuit 602 is illustrated to show the traces 404 prior to dicing (at 606) and after dicing (at 608).

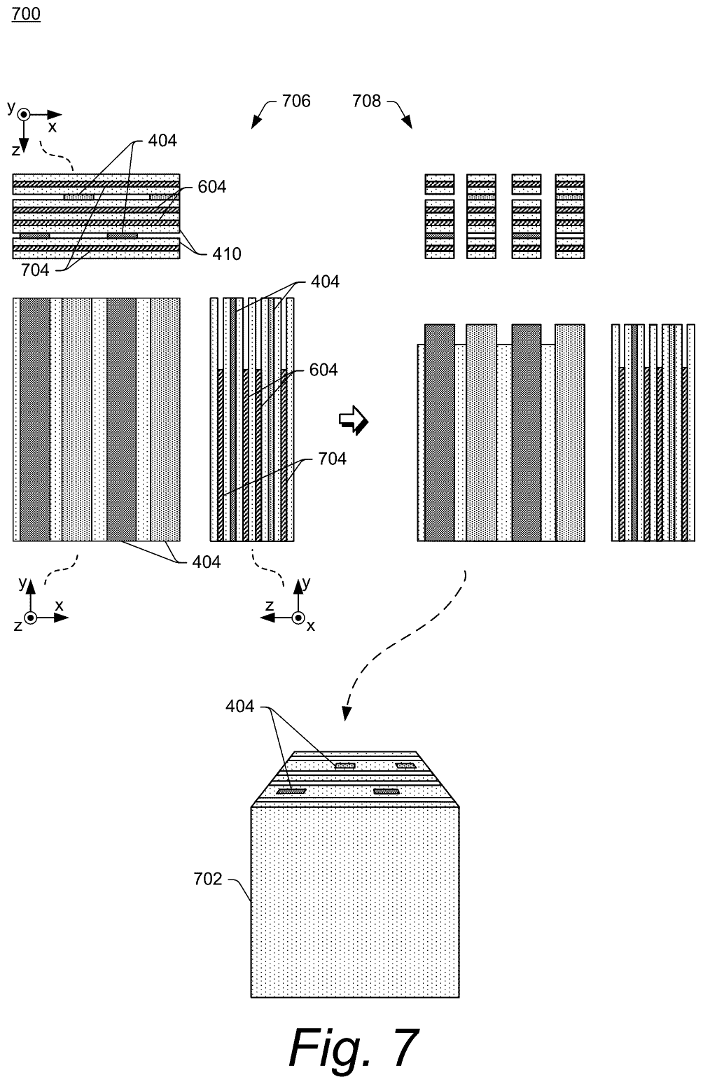

FIG. 7 illustrates an example implementation 700 of a multi-layer flex circuit 702 with internal and external ground planes 604 and 704, respectively. For simplicity, the multi-layer flex circuit 702 is illustrated to show the traces 404 prior to dicing (at 706) and after dicing (at 708). The external ground planes 704 (e.g., external ground layers) are disposed such that the traces 404 are between at least two of the external ground planes 704. The internal ground planes 604 are disposed between the traces 404. In the illustrated example, each trace layer is stacked between two ground planes (e.g., the internal ground plane 604 and the external ground plane 704). For example, the internal ground plane 604 and the external ground plane 704 are adjacent to opposing sides of the trace 404, with a first insulating layer 410 separating the trace 404 from the internal ground plane 604 and a second insulating layer 410 separating the trace 404 from the external ground plane 704.

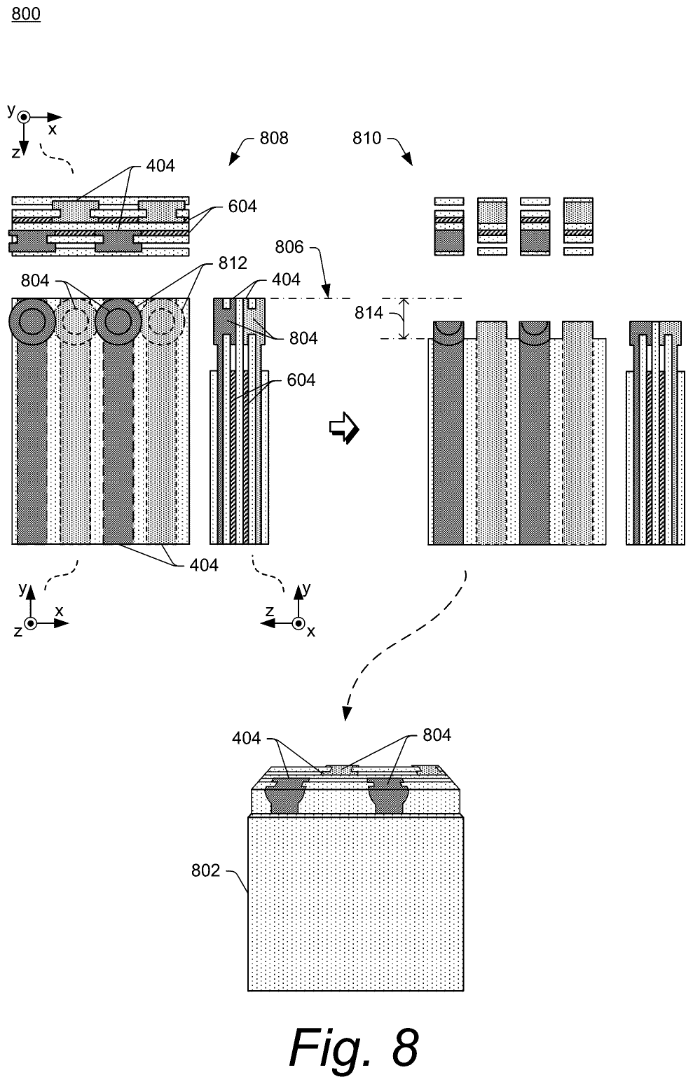

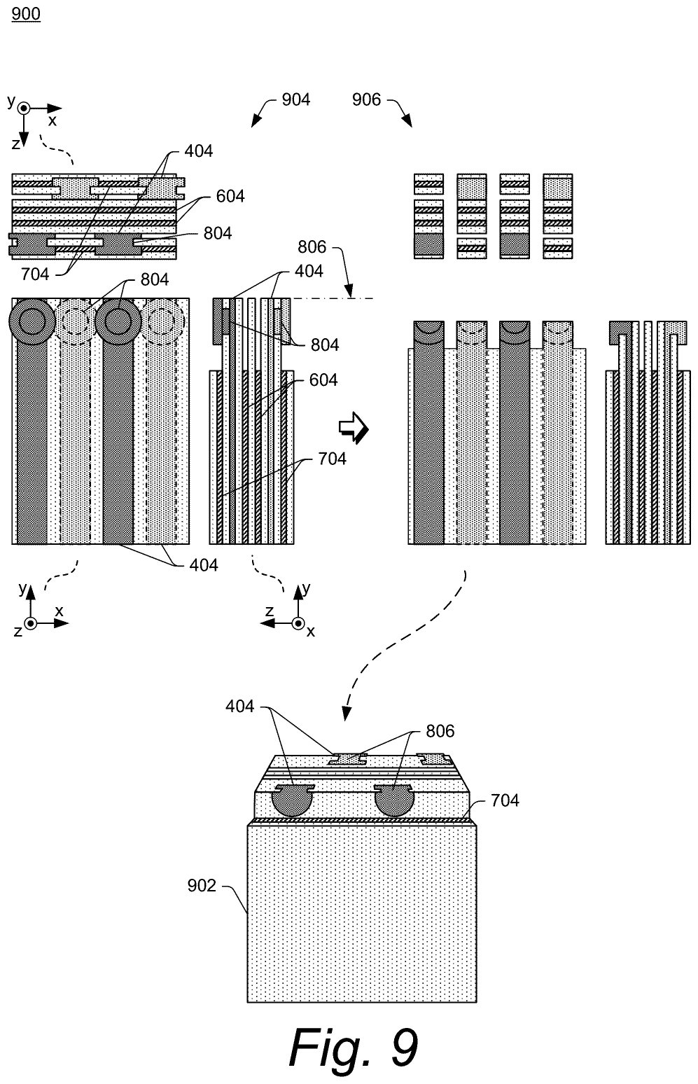

FIG. 8 illustrates and example implementation 800 of a multi-layer flex circuit 802 with internal ground planes 604 and plated-and-filled micro-vias 804 at a machining interface 806. A via 804 can connect a trace 404 in a first layer of the multi-layer circuit 802 to a second layer of the multi-layer circuit 802. The second layer can correspond to a ground layer, such as an internal ground plane 604 or an external ground plane 704, without connecting the trace 404 to ground. Another via 804 can connect a trace 404 in a third layer of the multi-layer circuit 802 to a fourth layer of the multi-layer circuit 802. The third layer can correspond to a ground layer, such as an internal ground plane 604 or an external ground plane 704, but the via 804 is not grounded (e.g., does not connect the trace 404 to ground). For simplicity, the multi-layer flex circuit 802 is illustrated to show the traces 404 prior to dicing (at 808) and after dicing (at 810). In the illustrated example, the vias 804 include an annular ring 812, which is also plated and filled. By comparing the multi-layer flex circuit 802 prior to dicing (at 808) and after dicing (at 810), it can be seen that the multi-layer flex circuit is cut to a depth (e.g., cut depth 814) that leaves the traces 404 exposed in a way that increases the surface area of conductive material and thereby increases the quality of electrical and physical connections to the traces 404. The examples illustrated in FIGS. 9-12 show similar cut depths for example only and are not intended to be limiting.

FIG. 9 illustrates an example implementation 900 of a multi-layer flex circuit 902 with internal and external ground planes 604 and 704, respectively, and plated-and-filled micro-vias 804 at the machining interface 806. For simplicity, the multi-layer flex circuit 902 is illustrated to show the traces 404 prior to dicing (at 904) and after dicing (at 906).

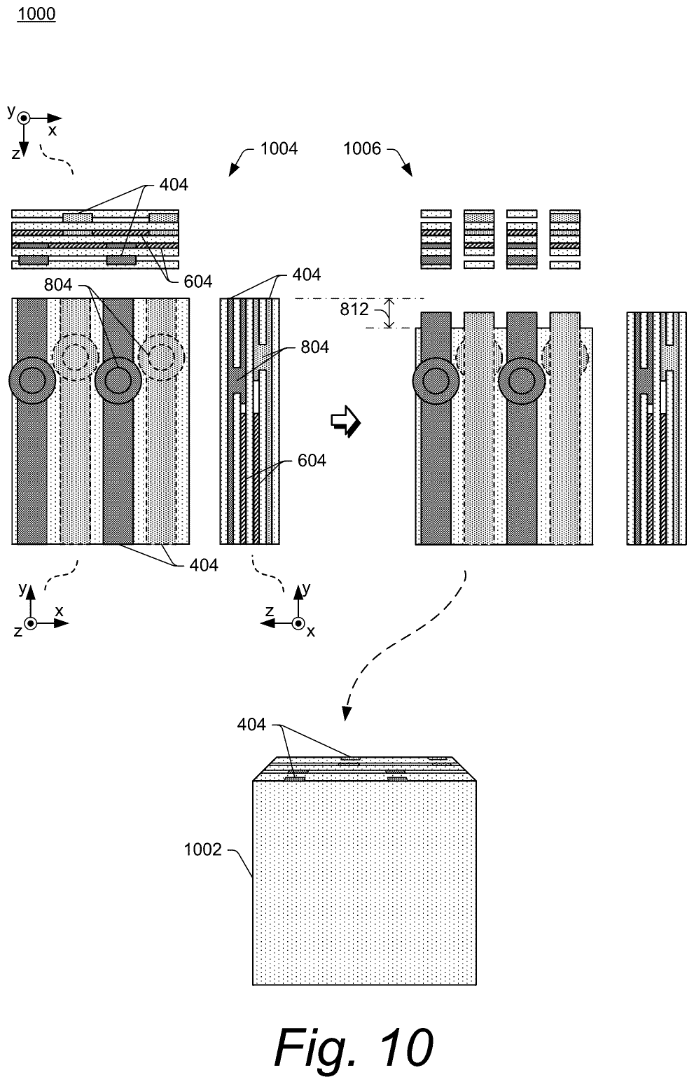

FIG. 10 illustrates an example implementation 1000 of a multi-layer flex circuit 1002 with internal ground planes 604 and plated-and-filled micro-vias 804 below the machining interface 806. For simplicity, the multi-layer flex circuit 1002 is illustrated to show the traces 404 prior to dicing (at 1004) and after dicing (at 1006).

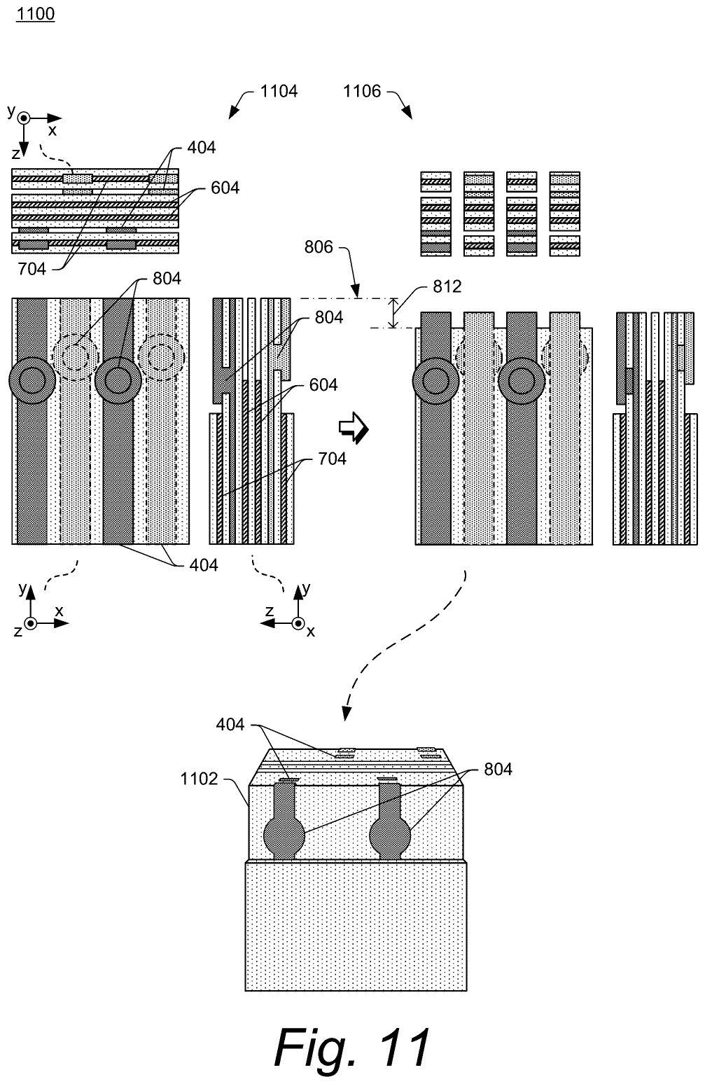

FIG. 11 illustrates an example implementation 1100 of a multi-layer flex circuit 1102 with internal and external ground planes 604 and 704, respectively, and plated-and-filled micro-vias 804 below the machining interface 806. For simplicity, the multi-layer flex circuit 1102 is illustrated to show the traces 404 prior to dicing (at 1104) and after dicing (at 1106).

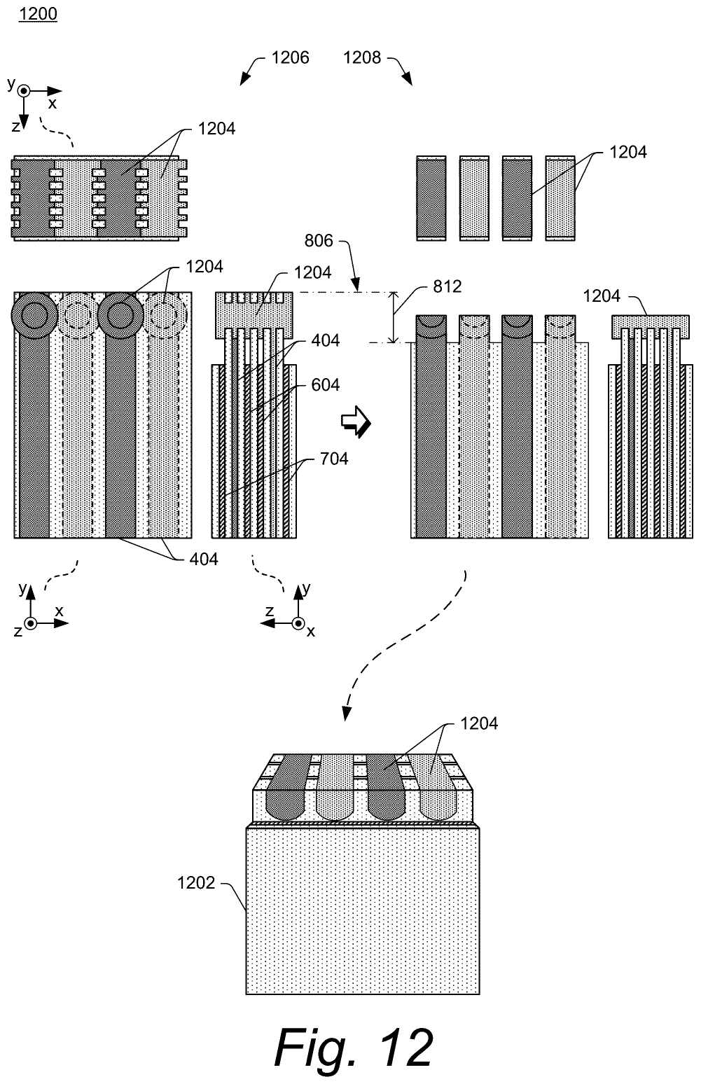

FIG. 12 illustrates an example implementation 1200 of a multi-layer flex circuit 1202 with internal and external ground planes 604 and 704, respectively, and plated-and-filled through-hole vias 1204 at the machining interface 806. For simplicity, the multi-layer flex circuit 1202 is illustrated to show the traces 404 prior to dicing (at 1206) and after dicing (at 1208).

A common failure point during transducer manufacturing happens when the electrical connection between the array block flex and the PZT matching layer stack fails. In many array block architectures, the surface area of the contact is limited to the width of a trace (˜100 μm) and the thickness of the copper (˜10 μm or about 1000 μm²). If a multilayer flex is used, there is an opportunity to add a filled via (e.g., via 804) to an adjacent layer or a layer that is disposed directly on an opposing side of an adjacent insulating layer. If this via is located at the location of the PZT interface (illustrated in FIGS. 8 and 9 where the via 804 is located at the machining interface 806), then after machining, the filled via can increase the surface area of the signal by a factor of ˜6×(100 μm×(10 μm (copper)+25 μm (Kapton)+10 μm (copper)+19 μm (plating))=6400 μm²). If the dicing depth is too deep to cut to the bottom of the via annular ring 812 at the via location, the vias can be shifted down below the cut depth (illustrated in FIGS. 10 and 11). In this case, the exposed surface area is increased by ˜4×(100 μm×(10 μm (copper)+10 μm (copper)+19 μm (plating))=3900 μm²). An additional trade off can be made between cross talk and contact surface area by extending the vias all the way through to short together with the neighboring trace. Then, the dicing can be relied on to separate the traces and leave the entire width of the flex as exposed copper for the electrical connection (see FIG. 12).

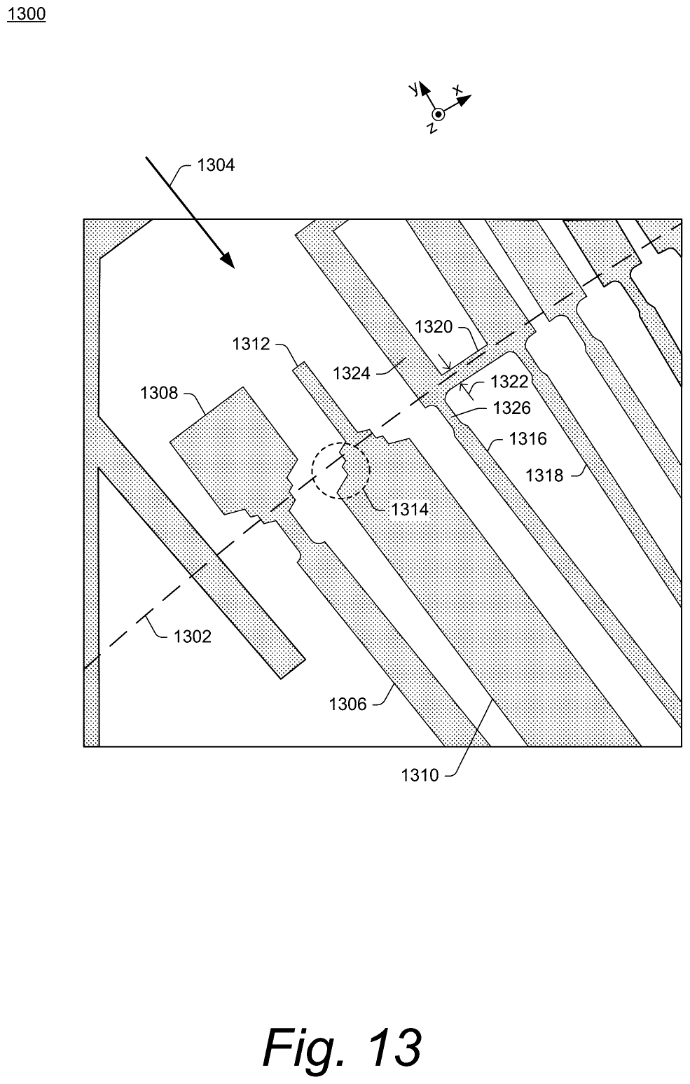

Alignment of the flex circuit in the backing block is key to high yield during machining. Since a significant portion of the flex circuit is discarded during the machining process, that material can be used to measure the accuracy of the alignment through either visual or electrical instrumentation. Some or all the layers can be used to create features that use resistance, capacitance, inductance, or optical measurements. For example, consider FIG. 13, which illustrates an example multi-layer flex circuit 1300 embedded in a backing block. The illustrated example provides a view of the flex circuit 1300 prior to machining and dicing.

Dashed line 1302 represents the nominal machined surface for the example flex circuit 1300. When machined, material above the dashed line 1302 is cut away (e.g., a part including the flex circuit 1300 is machined (e.g., milled) away in the direction of arrow 1304). The portion of the flex circuit 1300 below the dashed line 1302 remains (e.g., the portion on the opposite side of the dashed line 1302 from the arrow 1304 remains). As illustrated, the flex circuit 1300 includes various example features that are usable to provide indications of machining accuracy (e.g., depth, alignment, etc.).

For example, a first trace 1306 includes or is connected to a large-width portion 1308 that tapers or steps down in width towards the dashed line 1302 in the direction of the arrow 1304. A second trace 1310 includes or is connected to a small-width portion that increases in width (e.g., tapering increase, stepwise increase) toward the dashed line 1302 in the direction of the arrow 1304. Monitoring the two portions (e.g., 1308 and 1312) of the two traces 1306 and 1310, respectively, during the machining process enables the operator to know the machining depth. For example, as the part is milled away, the large-width portion 1308 decreases in width and the small-width portion 1312 increases in width.

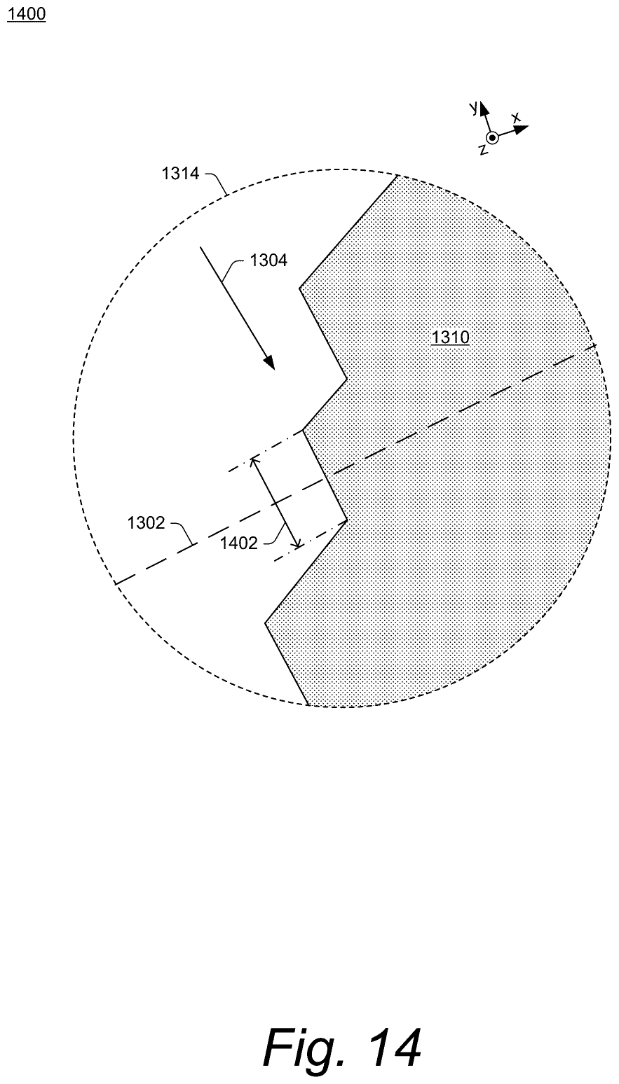

The portions 1308 and 1312 can be visibly observed and changes to one or more physical dimensions (e.g., length, width) can be monitored as the flex circuit is milled away. For example, if the two portions 1308 and 1312 are not the same size, then measuring them can provide an estimation where the current cut is from nominal (e.g., nominal depth is at dashed line 1302). In some implementations, if the outside trace (e.g., trace 1306) is larger than the inside trace (e.g., trace 1310), then the machining radius is too large. If the outside trace (e.g., trace 1306) is smaller than the inside trace (e.g., trace 1310), then the machining radius is too small. As the flex circuit 1300 is milled away, at some point within a tolerance depth of the dashed line 1302, the widths of the two portions 1308 and 1312 will be substantially equal to one another, which provides a visual indication that the correct depth has been reached for the machining process on the part. Further, if the width of the first trace 1306 becomes narrower than the width of the second trace 1310, then the operator can know that too much material has been machined away. Section 1314 is shown in an enlarged view in FIG. 14.

FIG. 14 illustrates an enlarged view 1400 of section 1314 from FIG. 13. As the part is machined away in the direction of arrow 1304, the width (x-dimension) of the second trace 1310, for example, can be measured and monitored. The second trace 1310 can have a predefined length (y-dimension) at the desired width that provides a tolerance for the machining process. The length (e.g., length 1402) can be any suitable length, such as a length within a range of 0.001 mil to 0.005 mils. For example, if the length 1402 is 0.002 mils, then the tolerance is +/−0.001 from center to have both of the traces 1306 and 1310 (shown in FIG. 13) be the same size (e.g., width).

Returning to FIG. 13, another feature implemented in the flex circuit to provide alignment aid includes connected traces, forming a sacrificial circuit. For example, traces 1316 and 1318 are connected by section 1320. A pad (not shown) can be connected to the outside trace (trace 1316) and used with a digital multimeter (DMM) to check the connection between that pad and a first pin on a connector. This connection check is for dicing, which is a major problem when the operator cannot tell if they have diced deep enough to clear the radius area (e.g., area 1322 of the section 1320 left by a cut in the direction of the arrow 1304) between the wide section (e.g., section 1324) of the trace 1316 and the narrow part (e.g., section 1326) of the trace 1316. In order to currently confirm there is no connection, using conventional techniques, the operator must pull the part off the dicing saw, run a capacitance test, and if there is a short, place the part back on the saw. Once the part is placed back on the saw, the part needs to be realigned but the part rarely gets aligned exactly using conventional techniques. However, the described implementation using the connected section 1320 can enable the operator to dice, then check and re-dice without removing the part from the saw. Accordingly, such a feature removes the need to realign the part, thereby reducing or eliminating misalignment issues on re-dice.

Further, if the flex circuit 1300 is diced at section 1320 (e.g., cut in the direction of the arrow 1304 to separate the traces 1316 and 1318) and the traces 1316 and 1318 are isolated and measured to confirm isolation, then such dicing provides an indication that the rest of the traces are also likely isolated. Accordingly, the section 1320 can enable on-the-fly monitoring without removing the part from the dicing machine, which can prevent the likelihood of error caused by re-setting the part in the dicing machine and making additional cuts.

The described features are on opposing sides (front and back) of the flex circuit. Carefully monitoring that these features on both sides of the flex circuit are even (the same) provides further aid during the machining process. In aspects, any suitable electrical characteristic that changes as portions of the part are removed during the machining process can be measured, including capacitance, impedance, resistance, etc.

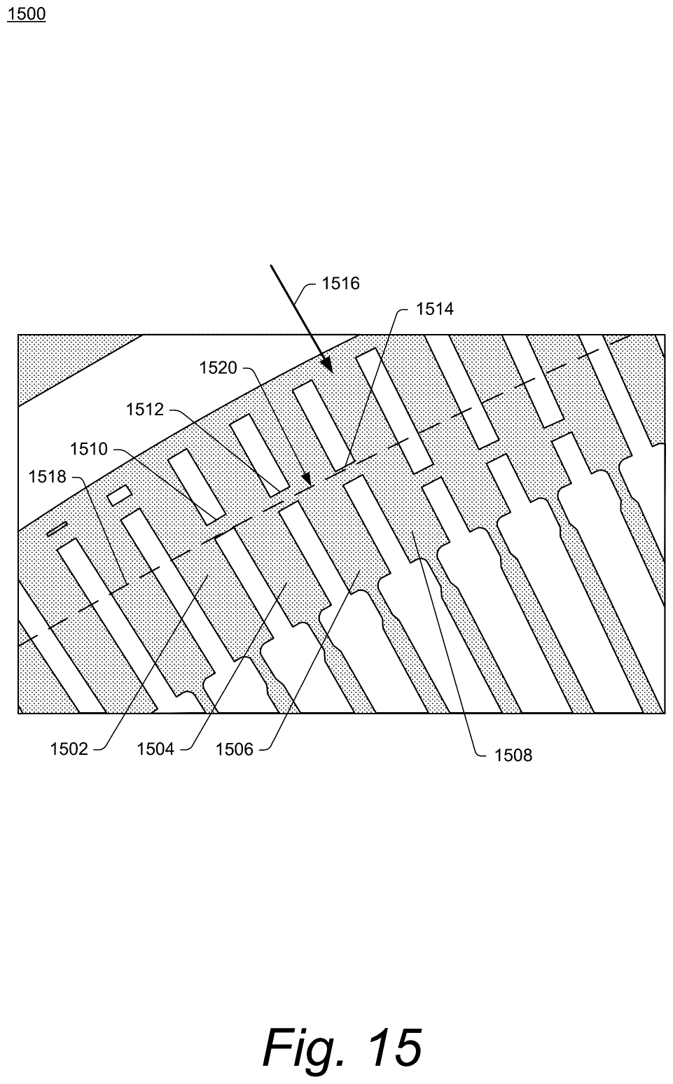

FIG. 15 illustrates an example flex circuit 1500 with additional features providing machining alignment aid. Another feature can be in three locations on the face of the array (e.g., left, center, and right sides). For example, as the part is machined down, the balance in the machining and the radius are checked. By so doing, the operator can catch unevenness and errors in the radius.

In an example, a printed circuit board (PCB) can have a light (e.g., light-emitting diode (LED)) electrically connected to one or more traces on the PCB. Several traces (e.g., traces 1502, 1504, 1506, and 1508) can be connected to one another by small connections (e.g., connections 1510, 1512, and 1514), respectively. Each connection between traces acts like a switch for one or more lights on the PCB. As the part is machined away in the direction of arrow 1516, one or more of the connections 1510, 1512, and 1514 are removed, affecting the electrical connection to the lights on the PCB. The dashed line 1518 represents an example machined surface 1520 as the part is machined away in the direction of the arrow 1516.

In an example in which the connections 1510, 1512, and 1514 are disposed at equal depths and each of the traces 1502, 1504, 1506, and 1508 is electrically connected to a different light, if all of the lights connected to the traces 1502, 1504, 1506, and 1508 turn off, then the simultaneous turning off of the lights indicates that the cut is even.

If the lights turn off at different times, then that indicates that the block is not being machined evenly. At least one of the connections 1510, 1512, and 1514 can be located at a depth that is different from a depth of the other little connections 1510, 1512, and 1514. In some implementations, each of the connections 1510, 1512, and 1514 are located at different depths compared to one another. Also, the connections are located at the same depths that the parts are typically machined at, so if the operator expects to machine the part so that only the last connection (e.g., connection 1514) is still connected, but the other two connections (e.g., connections 1510 and 1512) are connected still, then the two connections provide an indication that a radius machining issue exists.

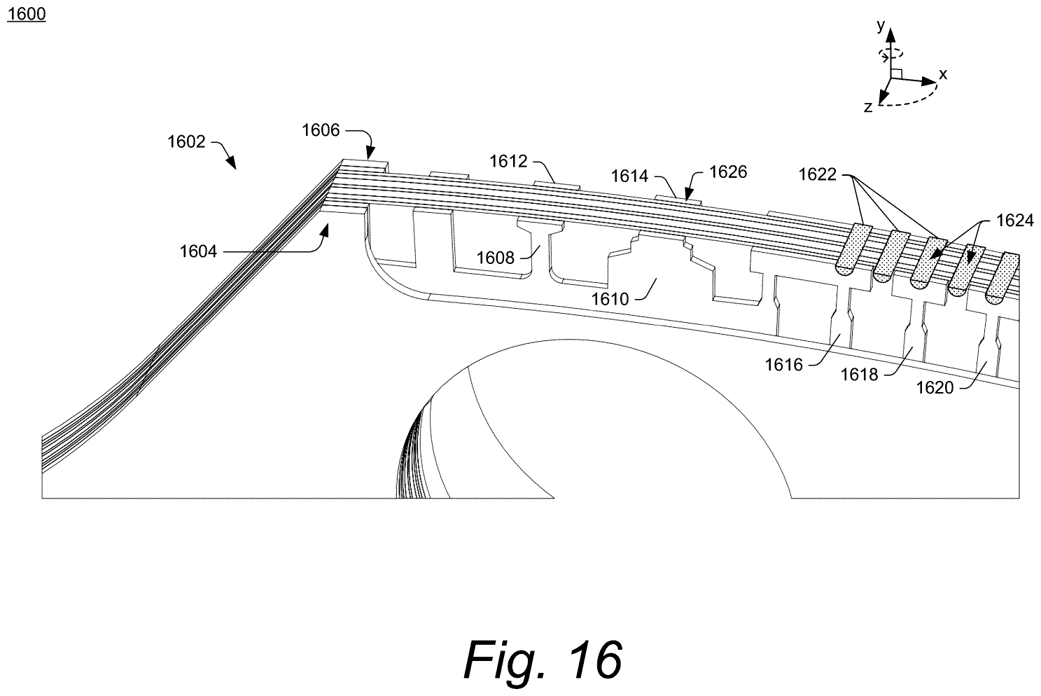

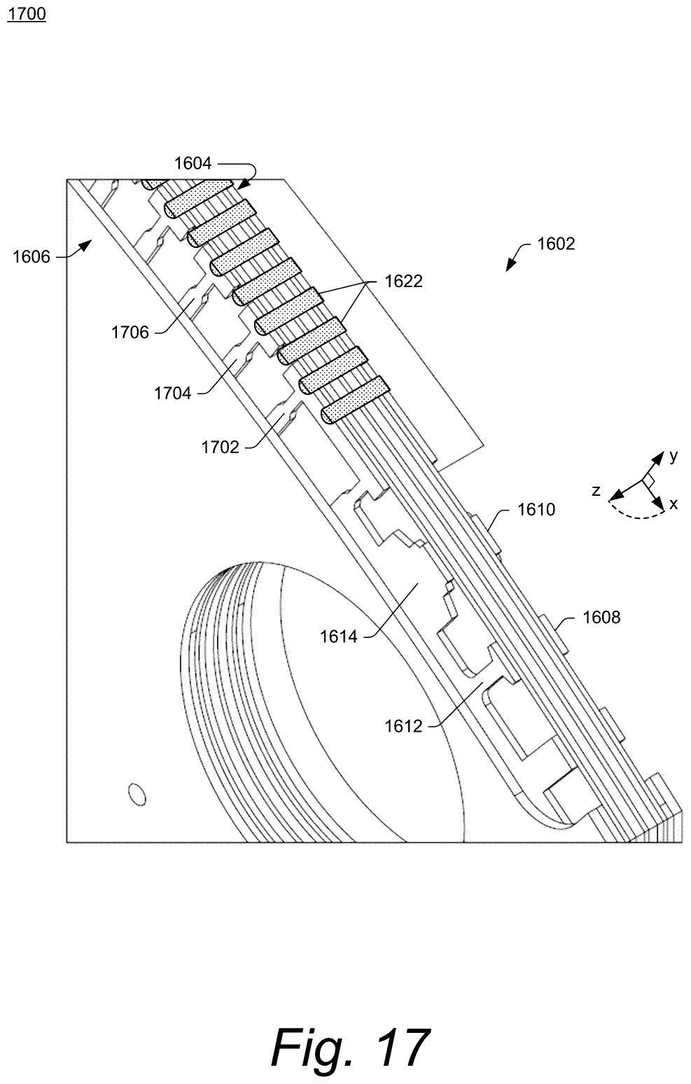

FIG. 16 illustrate an example implementation 1600 of a multi-layer flex circuit 1602 having multiple machining alignment aids. FIG. 17 illustrates another view 1700 of the multi-layer flex circuit 1602 from FIG. 16. FIG. 16 depicts a front side 1604 (orthogonal to the z-axis) of the multi-layer flex circuit 1602. FIG. 17 depicts a back side 1606 (orthogonal to the z-axis) of the multi-layer circuit 1602, which is opposite the front side 1604. The illustrated circuit 1602 is shown post-machining. Portions of some of the machining alignment aids used during the machining process can be seen on opposing sides (orthogonal to the z-axis) of the illustrated circuit 1602, such as from and back sides 1604 and 1606, respectively. For example, neighboring traces 1608 and 1610 having widths that change inversely with respect to one another below top surfaces (machined surfaces) that have equal widths (similar to traces 1306 and 1310 in FIG. 13). Such a combination of features indicates that the desired depth was reached during machining. Also, corresponding traces 1612 and 1614 on the back side of the circuit 1602 have matching widths, which indicates that the machined surface was cut evenly.

In addition, some traces (e.g., traces 1616, 1618, and 1620) have filled vias 1622 that have been partially machined to create a larger connection surface 1624 compared to just the end of the trace being the connection surface. For example, the connection surface 1624 formed from the filled vias 1622 is larger than a connection surface 1626 of the trace 1614. In the illustrated example, the vias 1622 extend through all the layers of the flex circuit 1602 but corresponding traces on the opposing side (e.g., traces 1702, 1704, and 1706 on the back side 1606 in FIG. 17) of the flex circuit 1602 are interleaved with the traces 1616, 1618, and 1620 on the front side (e.g., odd and even traces). Thus, the vias 1622 do not connect traces on opposing sides of the flex circuit to each other.

### Example Methods

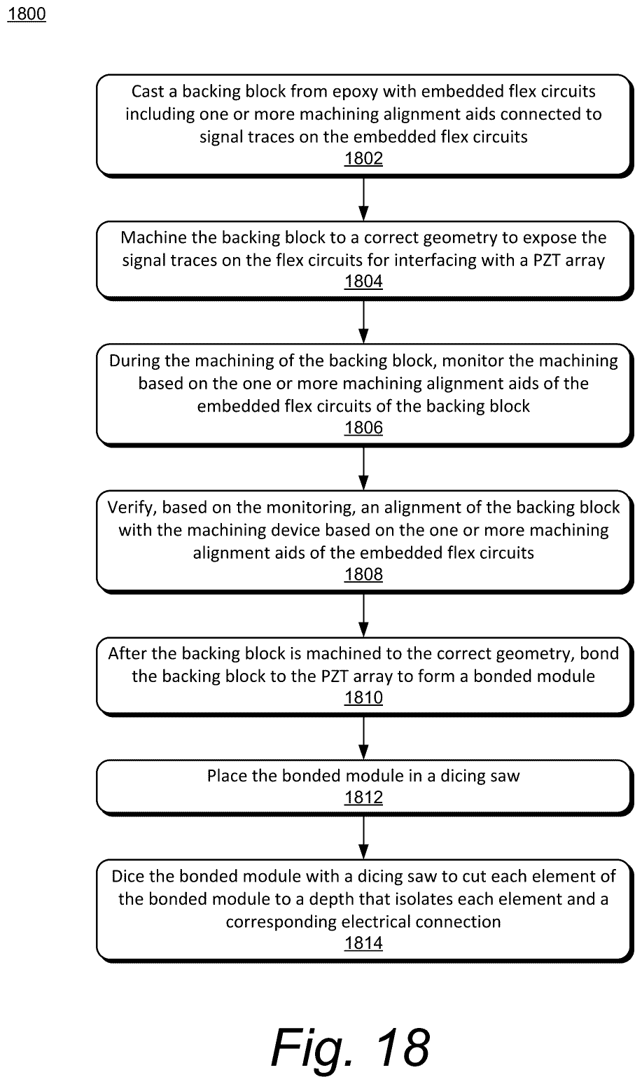

FIG. 18 depicts a method 1800 of manufacturing a flex circuit in accordance with the implementations disclosed herein. The method 1800 is shown as a set of blocks that specify operations performed but are not necessarily limited to the order or combinations shown for performing the operations by the respective blocks. Further, any of one or more of the operations can be repeated, combined, reorganized, or linked to provide a wide array of additional and/or alternate methods. In portions of the following discussion, reference can be made to the example system 100 of FIG. 1 or to entities or processes as detailed in FIGS. 2-17, reference to which is made for example only. The techniques are not limited to performance by one entity or multiple entities operating on one device.

At 1802, a backing block is cast from epoxy with embedded flex circuits including one or more machining alignment aids connected to signal traces on the embedded flex circuits. The machining alignment aids can be portions of the signal traces as described with respect to FIGS. 13-17, such as portions 1308 and 1312, sections 1320 and 1324, connections 1510, 1512, and 1514, etc.

At 1804, the backing block is machined to a correct geometry to expose the signal traces on the flex circuits for interfacing with a lead zirconate titanate (PZT) array. In aspects, a milling machine can be used to remove a portion of the backing block. For example, a portion of the part described in FIGS. 13 and 14 is removed in the direction of the arrow 1304 toward a target depth represented by the dashed line 1302. In another example, a portion of the part described in FIG. 15 is removed in the direction of the arrow 1516 toward a target depth represented by the dashed line 1518.

At 1806, during the machining of the backing block, the machining is monitored based on the one or more machining alignment aids of the embedded flex circuits of the backing block. In one example, in FIG. 13, as the part is milled away, the large-width portion 1308 decreases in width and the small-width portion 1312 increases in width. Changes to the physical dimensions of the portions 1308 and 1312 can be visibly observed and measured to provide an estimation of the current cut depth relative to the nominal depth (e.g., target depth). Other examples of the machining alignment aids being used for monitoring the machining are described with respect to FIGS. 13-17.

At 1808, based on the monitoring, an alignment of the backing block is verified with the machining device based on the one or more machining alignment aids of the embedded flex circuits. For example, if a first trace (e.g., trace 1306) is, at the machining interface, substantially the same size as a second, adjacent trace (e.g., trace 1310), then the correct depth has been reached for the machining process. If the first trace is smaller than the second trace, then the machining radius is too small. If the first trace is larger than the second trace, then the machining radius is too large. Other examples are described with respect to FIGS. 13-17.

At 1810, after the backing block is machined to the correct geometry, the backing block is bonded to the PZT array to form a bonded module. The backing block can be bonded to the PZT array using any suitable bonding technique.

At 1812, the bonded module is placed in a dicing saw. The bonded module is set and aligned with the dicing saw to enable the bonded module to be cut to a depth that isolates each element and its electrical connection.

At 1814, the bonded module is diced with a dicing saw to cut each element of the bonded module to a depth that isolates each element and a corresponding electrical connection. Examples of circuits prior to dicing and after dicing are comparatively illustrated and described with respect to FIGS. 4A-12.

While the examples described herein use flexible circuits, the disclosed techniques can also be implemented using a hard PCB. Further, although the examples described herein use a PZT ultrasound transducer, these techniques can be implemented using any suitable electromechanical array, sensor, or transducer, which uses an electrical connection from one structure to another structure.

### CONCLUSION

Embodiments of a multi-layer flexible array interconnect for an ultrasound transducer, and methods of manufacture described herein are advantageous, as they can provide more-consistent and controllable alignment between odd and even flex layers, improve crosstalk between signal layers, and simplify construction of flex circuits. In addition, the disclosed techniques can increase the surface area of conductive traces and/or reduce the amount of scrap material.

What is claimed is:

1. A flex array interconnect for an ultrasound circuit, the flex array interconnect comprising:
a first signal layer having a first plurality of conductive traces configured to be electrically and physically connected to an interface of an electromechanical-array element of an ultrasound transducer;
a second signal layer having a second plurality of conductive traces configured to be electrically and physically connected to the interface of the electromechanical-array element of the ultrasound transducer; and
one or more internal ground layers disposed between the first and second signal layers to reduce crosstalk between the first and second signal layers.

2. The flex array interconnect of claim 1, further comprising at least two external ground layers disposed such that the first and second signal layers are between the at least two external ground layers.

3. The flex array interconnect of claim 2, wherein each of the first and second signal layers is between the one or more internal ground layers and an external ground layer of the at least two external ground layers.

4. The flex array interconnect of claim 1, further comprising a plurality of vias that are filled and plated with conductive material, wherein:
a first via of the plurality of vias connects a first conductive trace of the first plurality of conductive traces in the first signal layer to a third layer of the flex array interconnect; and
the first via provides an enlarged surface area for the first conductive trace at a machining interface of the ultrasound circuit, in comparison to a cross section of the first conductive trace, for connection to the interface of the electromechanical-array element of the ultrasound transducer.

5. The flex array interconnect of claim 4, wherein the third layer corresponds to one of the internal ground layers and the first via is not grounded.

6. The flex array interconnect of claim 4, wherein the third layer corresponds to an external ground layer and the first via is not grounded, the first signal layer disposed between the external ground layer and the one or more internal ground layers.

7. The flex array interconnect of claim 4, wherein:
a second via of the plurality of vias connects a second conductive trace of the second plurality of conductive traces in the second signal layer to a fourth layer of the flex array interconnect; and
the second via provides an enlarged surface area for the second conductive trace at the machining interface of the ultrasound circuit, in comparison to a cross section of the second conductive trace, for connection to the interface of the electromechanical-array element of the ultrasound transducer.

8. The flex array interconnect of claim 7, wherein:
the one or more internal ground layers include first and second internal ground layers;
the third layer corresponds to the first internal ground layer and the first via is not grounded; and
the fourth layer corresponds to the second internal ground layer and the second via is not grounded.

9. The flex array interconnect of claim 7, wherein:
the third layer corresponds to a first external ground layer and the first via is not grounded;
the first signal layer is disposed between the first external ground layer and the one or more internal ground layers;
the fourth layer corresponds to a second external ground layer and the second via is not grounded; and
the second signal layer disposed between the second external ground layer and the one or more internal ground layers.

10. An ultrasound circuit comprising:
a plurality of layers including at least a first layer and a second layer, the first layer having a first plurality of conductive traces, the second layer having a second plurality of conductive traces; and
a plurality of vias that are filled and plated with conductive material, a first via of the plurality of vias connecting a first trace of the first plurality of traces in the first layer to a third layer of the plurality of layers, the first via providing an enlarged surface area for the first trace at a machining interface of the ultrasound circuit, in comparison to a cross section of the first trace, for connection to an interface of an electromechanical-array element of an ultrasound transducer.

11. The ultrasound circuit of claim 10, wherein a second via of the plurality of vias connects a second trace of the plurality of traces in the second layer to a fourth layer of the plurality of layers, the second via providing an enlarged surface area for the second trace at the machining interface of the ultrasound circuit, in comparison to a cross section of the second trace, for connection to the interface of the electromechanical-array element of the ultrasound transducer.

12. The ultrasound circuit of claim 11, wherein:
the second layer is disposed between the first layer and the third layer such that the first via extends through the second layer from the first layer to the third layer; and
the third layer is disposed between the first layer is between the second layer and the fourth layer such that the second via extends through the first layer from the second layer to the fourth layer.

13. The ultrasound circuit of claim 10, further comprising:
first and second external ground layers disposed on opposing sides of the ultrasound circuit such that the first layer and the second layer are both between the first and second external ground layers.

14. The ultrasound circuit of claim 13, wherein the first via and the second via each extend through the first and second layers from the first external ground layer to the second external ground layer, wherein the first and second vias are not grounded.

15. A flex array interconnect for an ultrasound circuit, the flex array interconnect comprising:
a first layer having a first plurality of conductive traces comprising a first set of machining alignment aids; and
a second layer stacked with the first layer and having a second plurality of conductive traces comprising a second set of machining alignment aids, the first and second sets of machining alignment aids providing measurable indicators of machining depth during a machining process.

16. The flex array interconnect of claim 15, wherein the measurable indicators are measurable based on at least one of capacitance, resistance, or impedance.

17. The flex array interconnect of claim 15, wherein the measurable indicators are visible and measurable based on one or more physical dimensions.

18. The flex array interconnect of claim 15, wherein the machining alignment aids provide measurable indicators of errors in a machining radius of the machining process.

19. The flex array interconnect of claim 15, wherein:
the first set of machining alignment aids includes a connection between two traces of the first plurality of conductive traces;
the connection electrically connects the two traces together until the connection is removed by the machining process; and
removal of the connection electrically isolates the two traces from one another, providing an indication of the machining depth.

20. The flex array interconnect of claim 19, further comprising one or more ground layers disposed between the first layer and the second layer to reduce crosstalk between the first plurality of conductive traces in the first layer and the second plurality of conductive traces in the second layer.
